(19) United States
(12) Plant Patent Application Publication
(10) Pub. No.: US 2003/0177549 P1
(43) Pub. Date: Sep. 18, 2003

(54) Miniature rose plant 'EVERgreen'

(76) Inventor: Rosa Eskelund Hansen, Faaborg (DK)

Correspondence Address: Rosa Eskelund Hansen, Fiskevaenget 9, Faaborg, 5600, DK

(21) Appl. No.: 10/098,209

(22) Filed: Mar. 18, 2002

(51) Int. Cl. A01H 5/00

(52) U.S. Cl. PLT/118

(57) ABSTRACT

A new miniature rose plant which has abundant, White-green coloured flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

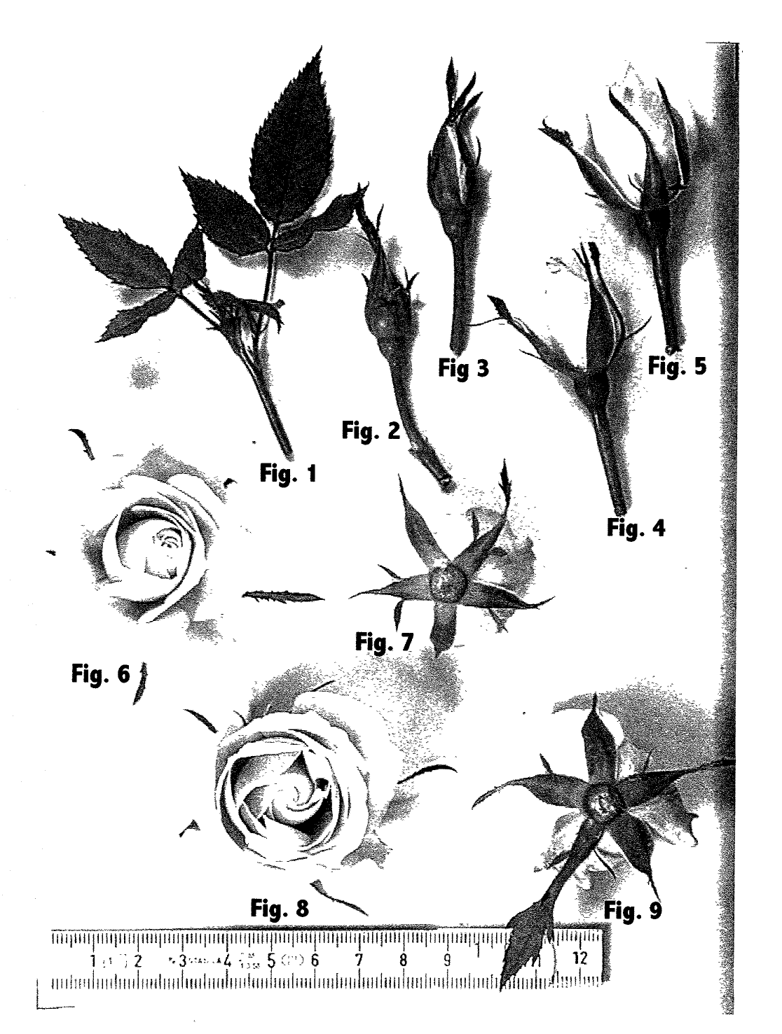

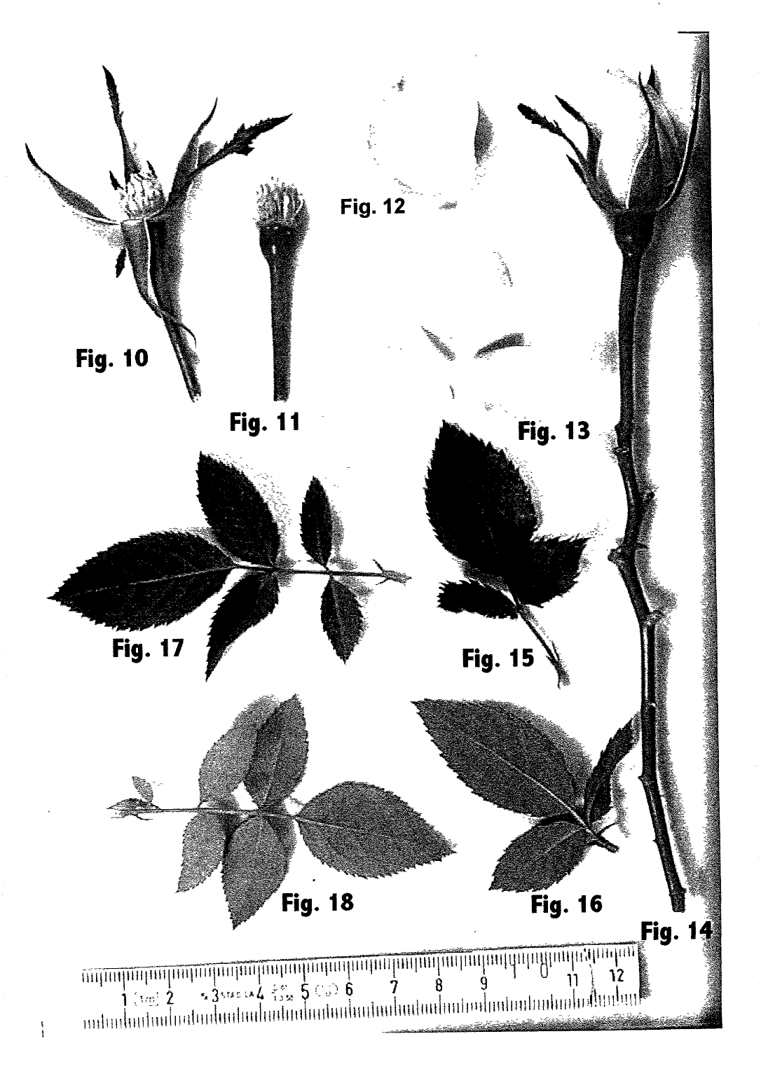

### BACKGROUND OF THE INVENTION

The present invention constitutes a new and distinct variety of miniature rose plant which was developed by artificially pollinating an unnamed seedling (not patent in the US) with an unnamed seedling (not patent in the US). The two parents were crossed in the summer of 2000 and the resulting seed was sown in December, 2001 in a controlled glasshouse environment. The new variety is named ‘EVERgreen’. The new rose may be distinguished from its seed parent, an unnamed seedling, by the following combination of characteristics:

1. The unnamed seedlings has a breeding background in unnamed seedlings.

2. ‘EVERgreen’ has big double flowers, while the unnamed seedling has big single flowers.

3. ‘EVERgreen’ has white-green coloured petals, while the unnamed seedling has pale yellow petals.

The new variety may distinguished from its pollen parent, an unnamed seedling created by the same inventor, by the following combination of characteristics:

1. The unnamed seedling has a breeding background in unnamed seedlings.

2. ‘EVERgreen’ has bigger flowers and foliage as compared to the unnamed seedling.

3. ‘EVERgreen’ has white-green coloured petals, while the unnamed seedling has light pink petals.

### BRIEF SUMMARY OF THE INVENTION

Asexual reproduction of ‘EVERgreen’ by cuttings was first done in Denmark and later in California, USA. The reproduction is done in controlled greenhouse environments. Have here proven to be stabile by propagation with cuttings in several generations. ‘EVERgreen’ is a low and compact miniature rose with good vigor. The shelflife has been tested to be excellent.

The objective of the hybridization of this rose variety for commercial greenhouse culture was to create a new and distinct variety with:

1. Uniform and abundant flowers with good keepability;

2. Attractive long lasting foliage and compact growth,

3. Year round flowering under glasshouse conditions;

4. Suitability for production from softwood cuttings in pots;

5. Durable flowers and foliage which make the variety suitable for distribution in the floral industry.

This combination of qualities was not present in previously available commercial cultivars of this type and distinguish ‘EVERgreen’ from all other varieties of which we are aware.

The seeds from hybridization were planted in a controlled environment and evaluations were conducted on the resulting plants. ‘EVERgreen’ was selected by Rosa Eskelund Hansen in her development program in Faaborg, Denmark.

### BRIEF DESCRIPTION OF THE DRAWINGS

The accompanying colour illustrations show as true as is reasonably to obtain in colour photographs of this type, the typical characteristics of the buds, flowers, leaves, stems of ‘EVERgreen’. Specifically illustrated in:

Photo sheet # 1:

1. Young shoot.

2. Bud before opening the sepals.

3. Bud at the opening the sepals.

4. Bud at the opening the petals.

5. Flower during course of opening.

6. Open flower—plan view—obverse.

7. Open flower—plan view—reverse.

8. Fully open flower- plan view—obverse.

9. Fully open flower—plan view—reverse.

Photo sheet # 2:

10. Receptacle showing stamens and pistils.

11. Receptacle showing pistils (stamens removed).

12. Flower petals, detached—outer surface.

13. Flower petals, detached—inner surface.

14. Bare stem exhibiting thorns and flower attachment.

15. 3 leaflets upper side.

16. 3 leaflets under side.

17. 5 leaflets upper side.

18. 5 leaflets under side.

### DETAILED BOTANICAL DESCRIPTION OF THE VARIETY

CLASSIFICATION:

Botanical.—Rosa hybrid, Commercial: Miniature The following is a detailed description of the Miniature Rose: Rosa hybrid ‘EVERgreen’, as observed in its growth in glass houses in Faaborg and AArhus, Denmark, and Glass-polyhouses in Santa Barbara California. The age of the observed plant was 11 to 13 weeks after propagation, produced as a flowering pot plant. The cultivation at both location was based on 20 to 25 dg. C. temperature. Descriptions were made from plants treated with growth regulators normally used in the greenhouse production process. The growth regulator Paclobutrazol was applied at 15-30 ppm. weekly beginning at a plant age of 8 weeks. The peduncle lengths mentioned may actually be sorter and the foliage colour several shades darker than on untreated specimens. Colour references are made using The Royal Horticultural Society (London, England) Colour Chart, 1995, except where common terms of colour are used. For a comparison, the nearest existing rose variety is ‘EVERgreen’, a rose variety described and illustrated in U.S. Plant Pat. No. PP,11,146. Chart 1 details several physical characteristics of ‘EVERgreen’ and ‘KORbritta’.

Chart 1: ‘EVERgreen’ ‘KORbritta’

Petal color, Upper surface Yellow Group 12D Yellow-Orange Group 21B

Petal color, Reverse surface Yellow Group 12D Yellow-Orange Group 21B

Petal count 30-35 20-25

Parents: Unnamed seeding. Times. Unnamed seedling

PLANT:

Plant growth.—Moderately vigorous. Grows compact upright to bushy. When grown as 10 cm pot plant, the average height of the plant itself is 18 to 20 cm, and average with is 20 cm. When grown as a 15 cm pot plant, the average height of the plant itself is 22 to 27 cm, and average with is 30 cm. Production time is generally 11 to 13 weeks depending on average temperature, light level, and cultural practices.

STEM:

Colour.—Young wood: Green Group 146A, with intonations of Greyed-Red Group 185A Older wood: Green Group 137A.

Thorns.—Incidence: Low number of thorns Size: 2-3 mm Colour: Greyed-Red Group 179D Shape: Deep concave.

Surface.—Young wood: Smooth Older wood: Smooth.

Stem diameter.—3-4 mm.

Internode length.—20-30 mm.

Numbers of internodes.—7-9.

PLANT FOLIAGE: Leaves arranged alternately, compound with three so seven leaflets per leaf, generally symmetrical, abundant, and flat in aspect. Stipules at petiole base.

Quantity of leaves.—Eight to nine per lateral branch.

Leaf size.—Medium 70-75 mm(l). times. 55-60 mm(w).

Petioles.—Colour.—Green Group 146A, with intonations of Greyed-Red Group 185A Margins: with small prickles. Length: 10-12 mm Diameter: about 1-2 mm.

Stipules.—Size: 5-6 mm Surface: Smooth Colour: Green Group 146B, with intonations of Greyed-Red Group 185A Margins: Serrated, single.

Rachis.—Colour: Green Group 146A, with intonations of Greyed-Red Group 185A Margins: Margins with small prickles. Length: 20 to 25 mm

Leaflets.—Edge: Serrated Serration: Double Shape: Ovate with acute apex and obtuse base Texture: Smooth Appearance: Glossy Size: length: 60 to 75 mm. With: 35 to 65 mm.

Colour.—Young foliage: Upper surface: Yellow-Green Group 146A With intonations of Greyed-Purple 183B Lower surface: Yellow-Green Group 147C, with intonations of Greyed-Purple Group 183D.

Colour.—Mature foliage: Upper surface: Green Group 136A Lower surface: Greyed-Green Group 191A.

INFLORESCENSE:

Blooming habit.—Recurrent.

Number of flowers.—Generally I bud per flowering stem.

Penduncle.—Colour: Yellow-Green Group 144A, Texture: Smooth, with stipitate glands Length: 35-45 mm Strength: Upright.

Receptacle.—Surface: Smooth, glabrous. Shape: Funnel-shaped Size: h: 6-7 mm w: 7-8 mm Colour: Yellow-Green Group 144A.

Sepals.—Quantity: 5 Shape: Narrowly Ovate w. acute tip Texture: Leathery Margin: Foliaceous appendages on 3 of the five sepals Appearance: Dull Colour: Upper surface: Yellow-Green Group 146B-C Reverse surface: Yellow-Green Group 138B-C.

Buds.—Size: 22-25 mm (h) 15-18 mm (w)upon opening. Shape: Pointed to cupped Colour: at ¼ opening, Yellow Group 12D, with intonations of Green-Yellow Group 149C at the edges, and Red Group 47C.

Flower.—

Duration.—As a pot plant, flowers last form 21 to 28 days. Fragrance. None. Size: 35-40 mm in diameter. Form: Shape of flower when viewed from the side. Up on opening: Cupped Open flower: Cupped Colour: Petals, upon opening. Upper surface: Yellow Group 12D, with intonations of Green-Yellow Group 149C. Reverse surface: Yellow-Green Group 149C, with intonations of Red Group 47C. Petals after opening: Upper surface: Yellow Group 12D, with intonations of Green-Yellow Group 149C edge. Reverse surface: Yellow-Green Group 149C, with intonations of Red Group 47C. Basal Petals spots: Size: 2-3 mm, Colour: Yellow-Green Group 154B General tonality: On Open flower: Third day: Greyed-Yellow Group 162B, with intonations of Yellow-Green 149C. Afterwards: Greyed-Yellow Group 162B, with intonations of Yellow-Green 149C.

Petals.—

Petal reflex.—Outermost petals reflex backwards at opening. Fully open all petals reflex backwards.

Texture.—Smooth.

Petal edge.—Uniform.

Petal count.—Approximately 30-35 on the average per flower.

Petal size.—Length 25 mm With: 30-35 mm.

Shape.—Outer petals: Round Inner petals: Ovate.

Petaloids.—Commonly none.

Reproductive organs.—

Stamen number.—Approximately 80-85 on average per flower.

Pollen.—Color: Red-Purple Group 59A, Abundance: Average.

Anthers.—Size: 1-2 mm Color: Yellow-Orange Group 22A, Shape: Oblong.

Filaments.—Size: 4-5 mm Colour: Yellow-Orange Group 14B.

Pistils number.—Approximately 40-45 on average per flower.

Stigmas.—Location: Superior in location to anthers Colour: Yellow-Orange Group 14B.

Styles.—Colour: Yellow-Orange Group 15C. Length: 5 to 6 mm.

DEVELOPMENT:

Vegetation.—Dense.

Blooming.—Abundant.

Aptitude to bear fruit.—Poor.

Resistance to diseases.—Above average resistance to mildew and Botrytis under normal growing conditions in AArhus, Denmark and Santa Barbara, Calif.

Hips/seeds.—Unknown, the plant have never been grown to the stage of seed development, due to the fact, that the variety is developed for use as a flowering potted plant only.

Winter hardiness & Drought/heat tolerance.—Due to the fact, that this variety is a potted flowering plant, developed for one time use only, the plant is not tested for winter hardiness or drought/heat tolerance.

1. A new and distinct variety of rose plant of the miniature class, substantially as herein illustrated and described as a distinct and novel rose variety due to its abundant White-green blend flowers, attractive long lasting foliage, vigorous and compact growth, year round flowering under glasshouse conditions, suitability for production from softwood cuttings in pots, and durable flowers and foliage which make the variety suitable for distribution in the floral industry.
